(19) United States
(12) Patent Application Publication
(10) Pub. No.: US 2024/0024166 A1
(43) Pub. Date: Jan. 25, 2024

(54) SHIELDABLE SWAB ASSEMBLY

(71) Applicant: Belal Hassanzadeh, Tabriz (IR)

(72) Inventor: Belal Hassanzadeh, Tabriz (IR)

(73) Assignee: Belal Hassanzadeh, Tabriz (IR)

(21) Appl. No.: 18/479,745

(22) Filed: Oct. 2, 2023

Related U.S. Application Data
(63) Continuation-in-part of application No. PCT/IB2022/053090, filed on Apr. 3, 2022.
(60) Provisional application No. 63/170,516, filed on Apr. 4, 2021.

(51) Int. Cl. A61F 13/38; A61B 10/00; A61F 13/551

(52) U.S. Cl. CPC A61F 13/38; A61B 10/0045; A61F 13/551

(57) ABSTRACT

A shieldable swab assembly comprising an elongated shaft, a collector tip, and a slidable cover assembly. The slidable cover assembly may comprise a hollow cover and a plurality of strings extending through a central duct of the elongated shaft. The elongated shaft may pass through proximal and distal apertures of the hollow cover. The hollow cover may be capable of moving along a longitudinal axis of the elongated shaft. Each respective string of the plurality of strings may comprise a distal end that may extend outwardly from a distal opening of the elongated shaft and may be attached to an outer surface of a distal end of the hollow cover. The each respective string of the plurality of strings may further comprise a proximal end that may extend outwardly from a proximal opening of the elongated shaft. Proximal ends of the plurality of strings may be tied to one another.

### CROSS-REFERENCE TO RELATED APPLICATION

This application is a continuation-in-part of PCT Application PCT/IB2022/053090, filed on Apr. 3, 2022, entitled “SHIELDABLE SWAB ASSEMBLY” which claims the benefit of priority from U.S Provisional Patent Application Ser. No. 63/170,516, filed on Apr. 4, 2021, entitled “SAMPLING SWAB HAVING SLIDABLE COVER ASSEMBLY”, which are both incorporated herein by reference in their entirety.

### TECHNICAL FIELD

The present disclosure generally relates to an exemplary shieldable swab assembly; in particular, the present disclosure relates to an exemplary shieldable swab assembly having a slidable cover assembly. The slidable cover assembly may protect a collector tip of the exemplary shieldable swab assembly from environmental and/or secondary contaminations pre-, post-, and during a sampling procedure and may decrease the risk of disease transmission.

### BACKGROUND

Sampling swabs, comprising an elongated shaft and a collector tip secured at one end, are common tools for collecting a wide variety of specimens including, but not limited to, organic/biological fluids, tissues, mucosa, environmental specimens, etc. Conventional sampling swabs may be prone to contamination prior to, during, and after conducting a sampling procedure; meanwhile, in some instances, conventional swabs may play a part in disease transmission.

Sample collection from target sites that are located through or at a depth of a canal may require customized swabs capable of passing through a canal and reaching a desired location. Thus, a collector tip that is responsible to collect sufficient specimen from a target site may be prone to touching sites surrounding a target site. For example, collection of oropharyngeal mucus may be accomplished by dipping collector tip of a sampling swab in a particular part of a subject's mouth without contacting other sites within the mouth. In this particular example, a sampling swab may be withdrawn, after smearing collector tip across throat and tonsils, without contacting collector tip with surrounding areas within subject's mouth. Exposure of a swab's collector tip to sites other than a target site may result in secondary and/or environmental contamination, and therefore may give rise to analytical errors. Furthermore, use of sampling swabs for collecting specimens of infectious and/or contagious diseases may increase the risk of disease transmission to healthcare personnel.

Furthermore, sampling swabs are disposable items and may be used in large quantities in healthcare centers and diagnostic laboratories. Thereby, there is need to produce low-priced sampling swabs that are protected against environmental and/or secondary contaminations pre-, post-, and during sampling procedure and decrease the risk of disease transmission.

### SUMMARY

This summary is intended to provide an overview of the subject matter of the present disclosure, and is not intended to identify essential elements or key elements of the subject matter, nor is it intended to be used to determine the scope of the claimed implementations. Its sole purpose is to present some concepts of one or more aspects in a simplified form as a prelude to the more detailed description that is presented later. The proper scope of the present disclosure may be ascertained from the claims set forth below in view of the detailed description below and the drawings.

One or more exemplary embodiments describe an exemplary shieldable swab assembly. In an exemplary embodiment, an exemplary shieldable swab assembly may comprise an exemplary elongated shaft and an exemplary collector tip secured around an exemplary distal end of an exemplary elongated shaft. In an exemplary embodiment, an exemplary elongated shaft may include an exemplary distal end comprising an exemplary distal opening, an exemplary proximal end comprising an exemplary proximal opening, and an exemplary central duct extending through an exemplary elongated shaft from distal end of an exemplary elongated shaft to proximal end of an exemplary elongated shaft.

In an exemplary embodiment, an exemplary shieldable swab assembly may further include an exemplary slidable cover assembly that may be carried by an exemplary elongated shaft. An exemplary slidable cover assembly may be configured to removably enclose an exemplary collector tip and may comprise an exemplary hollow cover made of an exemplary resilient material and exemplary plurality of strings extending through an exemplary central duct of an exemplary elongated shaft. In an exemplary embodiment, an exemplary hollow cover may have an exemplary proximal end that may be slidably fastened around an exemplary elongated shaft on an exemplary rear side of an exemplary collector tip. In an exemplary embodiment, an exemplary hollow cover may further include an exemplary distal end. In an exemplary embodiment, an exemplary distal end of an exemplary hollow cover may comprise an exemplary distal aperture that may adopt an exemplary closed configuration and an exemplary open configuration. In an exemplary embodiment, an exemplary elongated shaft may pass through exemplary proximal and distal apertures of an exemplary hollow cover. In an exemplary embodiment, an exemplary hollow cover may be capable of moving along an exemplary longitudinal axis of an exemplary elongated shaft toward exemplary proximal and distal ends of an exemplary elongated shaft.

In an exemplary embodiment, an exemplary slidable cover assembly may comprise a plurality of exemplary strings extending throughout an exemplary central duct of an exemplary elongated shaft. In an exemplary embodiment, each respective string of exemplary plurality of strings may comprise an exemplary distal end extending outwardly from an exemplary distal opening of an exemplary elongated shaft and an exemplary proximal end extending outwardly from an exemplary proximal opening of an exemplary elongated shaft. In an exemplary embodiment, an exemplary distal end of each respective string may be attached to an outer surface of an exemplary distal end of an exemplary hollow cover. In an exemplary embodiment, distal ends of exemplary plurality of strings may be arranged radially and concentrically around an exemplary distal end of an exemplary hollow cover. In an exemplary embodiment, exemplary proximal ends of exemplary plurality of strings may be tied to one another.

In an exemplary embodiment, an exemplary hollow cover may be configured to adopt an exemplary first configuration in a first state in which an exemplary hollow cover may enclose an exemplary collector tip. In an exemplary embodiment, in an exemplary first configuration, an exemplary distal aperture of an exemplary hollow cover may adopt an exemplary closed configuration in which an exemplary distal aperture of an exemplary hollow cover may be closed around an exemplary distal end of an exemplary collector tip.

In an exemplary embodiment, an exemplary hollow cover may further be configured to adopt an exemplary second configuration in a second state when an exemplary hollow cover is moved along an exemplary longitudinal axis of an exemplary elongated shaft toward an exemplary proximal end of an exemplary elongated shaft such that an exemplary distal aperture of an exemplary hollow cover opens and an exemplary collector tip or a portion of an exemplary collector tip becomes uncovered.

In an exemplary embodiment, an exemplary hollow cover may further be configured to adopt an exemplary third configuration in an third state when exemplary proximal ends of exemplary plurality of strings are pulled away from an exemplary proximal end of an exemplary elongated shaft such that an exemplary hollow cover when in an exemplary second state is retracted toward an exemplary distal end of an exemplary elongated shaft and may enclose an exemplary collector tip.

This Summary may introduce a number of concepts in a simplified format; the concepts are further disclosed within the “Detailed Description” section. This Summary is not intended to configure essential/key features of the claimed subject matter, nor is intended to limit the scope of the claimed subject matter.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which a presently preferred embodiment of the present disclosure will now be illustrated by way of example. It is expressly understood, however, that the drawings are for the purpose of illustration and description only and are not intended as a definition of the limits of the present disclosure. Embodiments of the present disclosure will now be described by way of example in association with the accompanying drawings in which:

FIG. 1A shows a schematic illustration of an exemplary shieldable swab assembly carrying an exemplary slidable cover assembly, consistent with one or more exemplary embodiments of the present disclosure;

FIG. 1B illustrates an exemplary shieldable swab assembly in a separated scenario, consistent with one or more exemplary embodiments of the present disclosure;

FIG. 1C schematically illustrates a cross-sectional view of an exemplary shieldable swab assembly carrying an exemplary slidable cover assembly, consistent with one or more exemplary embodiments of the present disclosure;

FIG. 2A shows a schematic illustration of an exemplary shieldable swab assembly in an exemplary first configuration and an exemplary second configuration, consistent with one or more exemplary embodiments of the present disclosure;

FIG. 2B shows a schematic illustration of an exemplary shieldable swab assembly in an exemplary third configuration, consistent with one or more exemplary embodiments of the present disclosure; and

FIG. 3 illustrates an exemplary method for sampling oropharyngeal mucosa using an exemplary shieldable swab assembly, consistent with one or more exemplary embodiments of the present disclosure.

### DETAILED DESCRIPTION

In the following detailed description, numerous specific details are set forth by way of examples to provide a thorough understanding of the relevant teachings related to the exemplary embodiments. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in one or more exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be plain to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Sampling swabs are widely used tools for collecting specimens of organic/biological fluids, tissues, and the like. In certain cases, a swab's collector tip may be inserted into a canal or an area to access an exemplary target site from which an exemplary sample is collected. Consequently, swab's collector tip may come into contact with adjacent areas surrounding an exemplary target site. For instance, acquiring cervical mucus may be accomplished by inserting a swab's collector tip into a subject's vaginal passage while avoiding contact with vaginal wall during swab insertion and removal. It is to be noted that inadvertent contact between a collector tip and adjacent areas other than an exemplary target site may give rise to secondary and/or environmental contamination of an exemplary specimen and, in turn, erroneous analytical results. “Secondary contamination” may refer to transfer of a contaminate from a contamination source to personnel or equipment.

Disclosed herein is an exemplary shieldable swab assembly. In an exemplary embodiment, an exemplary shieldable swab assembly may comprise an exemplary slidable cover assembly which may aid in protecting an exemplary collector tip of an exemplary shieldable swab assembly from contacting with adjacent areas surrounding an exemplary target site. “Target site” or “sampling site” may refer to any location or site from which a specimen is collected.

In an exemplary embodiment, an exemplary slidable cover assembly may protect an exemplary collector tip from secondary contamination, environmental contamination, an/or cross-contamination pre, post, or during a sampling procedure. Meanwhile, an exemplary shieldable swab assembly may decrease risk of disease transmission to healthcare personnel/operator. In an exemplary embodiment, an exemplary shieldable swab assembly may be used for rectal wall sampling, oropharyngeal sampling, nasopharyngeal sampling, nasal sampling, oral sampling, laryngeal sampling, etc. Contact of a collector tip to surrounding sites other than an exemplary target site may result in secondary and/or environmental contamination of a sample and, therefore, erroneous analytical results.

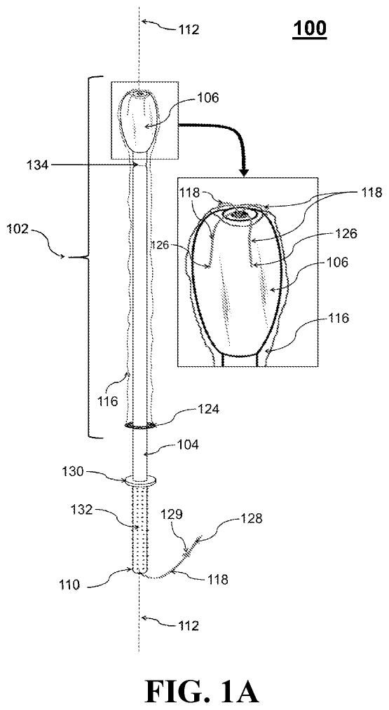

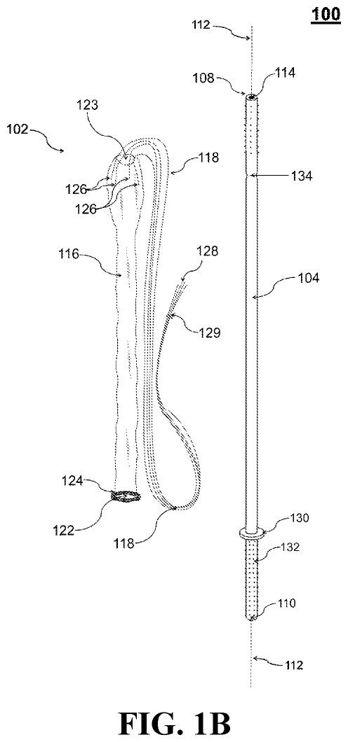

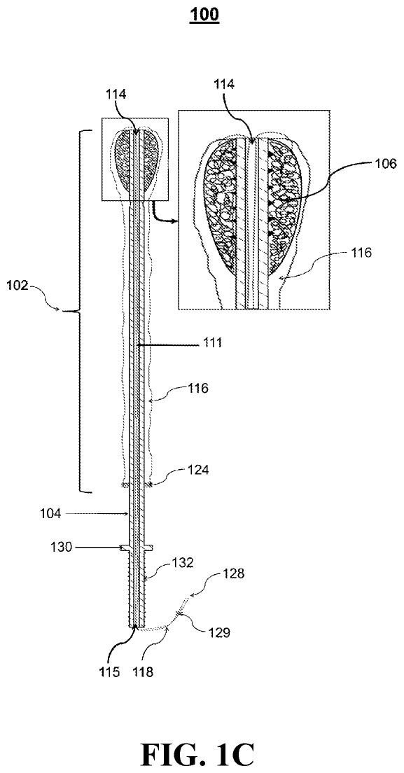

FIGS. 1A-C illustrate exemplary embodiments of an exemplary shieldable swab assembly. More particularly, FIG. 1A shows a schematic illustration of exemplary shieldable swab assembly 100 carrying slidable cover assembly 102, consistent with one or more exemplary embodiment of the present disclosure. FIG. 1B illustrates exemplary shieldable swab assembly 100 in a separated scenario, consistent with one or more exemplary embodiments of the present disclosure. FIG. 1C schematically illustrates a cross-sectional view of exemplary shieldable swab assembly 100 carrying slidable cover assembly 102, consistent with one or more exemplary embodiments of the present disclosure. In further detail with respect to FIGS. 1A-C, in an exemplary embodiment, exemplary shieldable swab assembly 100 may include elongated shaft 104, collector tip 106 secured around distal end 108 of elongated shaft 104, and slidable cover assembly 102. In an exemplary embodiment, elongated shaft 104 may include distal end 108, proximal end 110, and central duct 111 (see FIG. 1C). In an exemplary embodiment, central duct 111 may extend through elongated shaft 104 along longitudinal axis 112 of elongated shaft 104 from distal end 108 toward proximal end 110 of elongated shaft 104. Distal end 108 may comprise distal opening 114, and proximal end 110 may comprise proximal opening 115. In an exemplary embodiment, exemplary shieldable swab assembly 100 may include slidable cover assembly 102 that may be carried by elongated shaft 104. Slidable cover assembly 102 may include hollow cover 116—configured to protect collector tip 106 from contamination (that is, due its structure, slidable cover assembly 116 may protect collector tip 106) by enclosing/covering collector tip 106)—and plurality of strings 118 extending throughout central duct 111 of elongated shaft 104. “Enclose,” “cover,” and the like may refer to a state in which something may be extended over an entire or at least a portion of an object, e.g., an exemplary collector tip of an exemplary shieldable swab assembly.

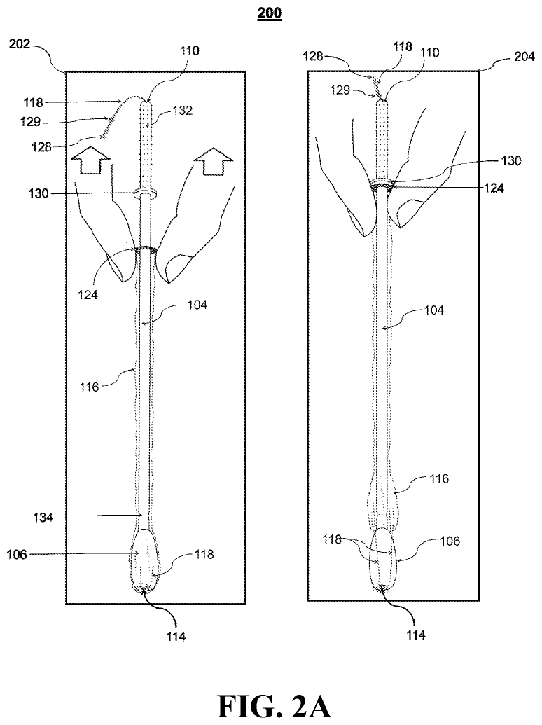

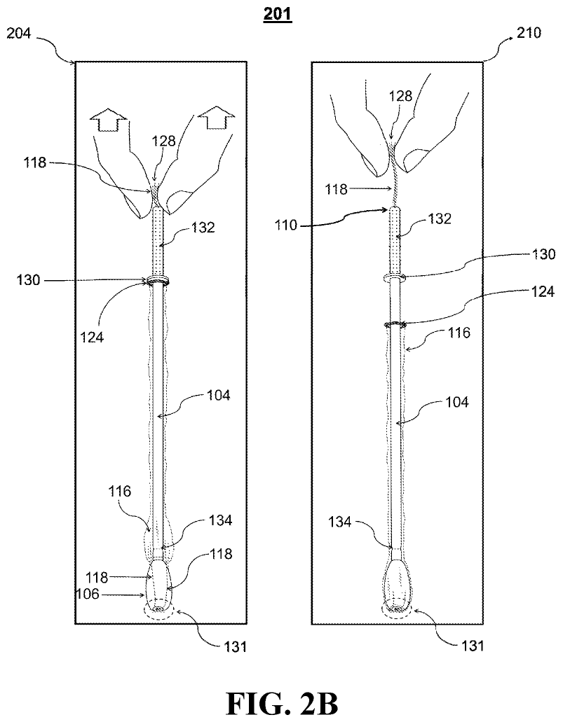

In an exemplary embodiment, hollow cover 116 may include proximal end 119 and distal end 120 (see FIG. 1B). In an exemplary embodiment, proximal end 119 of hollow cover 116 may be slidably fastened around elongated shaft 104 on a rear side of collector tip 106. Elongated shaft 104 may pass through proximal and distal apertures (122 and 123, respectively) of hollow cover 116 such that hollow cover 116 may be capable of sliding on elongated shaft 104, along longitudinal axis 112 of elongated shaft 104 toward proximal and distal ends (110 and 108, respectively) of elongated shaft 104. In an exemplary embodiment, proximal end 119 of hollow cover 116 may be slidably fastened around elongated shaft 104 using band 124 (such as a grommet, a washer and the like) that may be twisted, perpendicular to longitudinal axis 112 of elongated shaft 104, around proximal end 119 of hollow cover 116. In an exemplary embodiment, band 124 may be an elastic band. In an exemplary embodiment, distal aperture 123 of hollow cover 116 may be capable of adopting an exemplary closed configuration and an exemplary open configuration that are illustrated in FIG. 2A and FIG. 2B (labeled as 206 and 208, respectively) and are described with further details below in context of descriptions of FIG. 2A and FIG. 2B.

In an exemplary embodiment, details of plurality of strings 118 of slidable cover assembly 102 are illustrated in context of elements presented in FIG. 1C. In particular, FIG. 1C illustrates how plurality of strings 118 are arranged inside central duct 111. In an exemplary embodiment, each respective string of plurality of strings 118 may have distal end 126 extending outwardly from distal opening 114 of elongated shaft 104, and proximal end 128 extending outwardly from proximal opening 115 of elongated shaft 104. In an exemplary embodiment, distal end 126 of each respective string of plurality of strings 118 may be attached to an outer surface of distal end 120 of hollow cover 116. In an exemplary embodiment, distal ends (e.g., distal end 126) of plurality of strings 118 may be arranged radially and concentrically around distal end 126 of hollow cover 116. In an exemplary embodiment, proximal ends (e.g., proximal end 128) of plurality of strings 118 may be tied to one another. In an exemplary embodiment, as illustrated in FIGS. 1A-C, proximal ends (e.g., proximal end 128) of plurality of strings 118 may be tied to one another by forming knot 129. In an exemplary embodiment, plurality of strings 118 may be used for moving hollow cover 116 along longitudinal axis 112 of elongated shaft 104, as described with further details below in context of descriptions of FIG. 2A and FIG. 2B.

In an exemplary embodiment, hollow cover 116 may adopt one or more exemplary configurations shown in FIG. 2A and FIG. 2B. FIG. 2A shows schematic illustration 200 of exemplary shieldable swab assembly 100 in first configuration 202 and second configuration 204, consistent with one or more exemplary embodiments of the present disclosure. In further detail with respect to FIG. 2A, in first configuration 202, hollow cover 116 may enclose collector tip 106 such that distal aperture 123 of hollow cover 116 adopts closed configuration 206. Closed configuration 206 may refer to an exemplary configuration in which distal aperture 123 of hollow cover 116 may be closed around distal end 210 of collector tip 106. In an exemplary embodiment, in closed configuration 206, distal end 120 of hollow cover 116 may be squeezed and fitted around collector tip 106. In an exemplary embodiment, diameter of distal aperture 123 in closed configuration 206 may be less than its diameter in open configuration 208.

With further reference to FIG. 2A, in an exemplary embodiment, hollow cover 116 may further adopt second configuration 204 that may be achieved by moving hollow cover 116 along longitudinal axis 112 of elongated shaft 104 toward proximal end 110 of elongated shaft 104, such that distal aperture 123 of hollow cover 116 opens (see open configuration 208 of distal aperture 123 in FIG. 2A) and collector tip 106 or a portion of collector tip 106 may become uncovered. For example, collector tip 106 may be uncovered to collect an exemplary specimen (e.g., oropharyngeal mucosa) or to dispense an exemplary collected specimen (e.g., oropharyngeal mucosa) on a physical substrate or in a liquid, such as an exemplary buffer or an exemplary growth medium (e.g., a microbial growth medium).

FIG. 2B shows schematic illustration 201 of exemplary shieldable swab assembly 100 in third configuration 210, consistent with one or more exemplary embodiments of the present disclosure. In further detail with respect to FIG. 2B, in an exemplary embodiment, hollow cover 116 (retaining second configuration 204) may further adopt third configuration 210 that may be achieved by pulling proximal ends (e.g., proximal end 128) of plurality of strings 118 away from proximal end 110 of elongated shaft 104 such that hollow cover 116—being in second configuration 204—may retract toward distal end 108 of elongated shaft 104 and may enclose collector tip 106 (of hollow cover 116 may become closed around distal end 131 (see FIG. 2B) of collector tip 106 (so that distal aperture 123 of hollow cover 116 may become closed around distal end 131 of collector tip 106). In an exemplary embodiment, when hollow cove 116 is in third configuration 210, distal aperture 123 of hollow cover 116 may adopt closed configuration 206.

With further reference to FIGS. 1A-C, in an exemplary embodiment, elongated shaft 104 may further comprise stopper 130 mounted on elongated shaft 104 further apart from proximal end 110 of elongated shaft 104 and on a rear side of proximal end 119 of hollow cover 116. Stopper 130 may be configured to prevent hollow cover 116 from sliding up to proximal end 110 of elongated shaft 104 and from being removed from elongated shaft 104, that is due to its position on a rear side of proximal end 119 of hollow cover 116. In further detail with respect to FIGS. 1A-C, in an exemplary embodiment, elongated shaft 104 may further include handle 132 that may circumscribe around proximal end 110 of elongated shaft 104 and further apart from proximal end 110 up to stopper 130. In an exemplary embodiment, a pathology collector may hold/carry exemplary shieldable swab assembly 100 in hand by gripping handle 132 between his/her fingers (e.g., thumb and index finger). “Pathology collector” may refer to a person who works for medical laboratories and may perform a range of tasks including, but not limited to, collecting, extracting, and labelling urine, blood, faeces, and saliva swabs and other patient samples.

In an exemplary embodiment, as shown in FIGS. 1A-C, elongated shaft 104 may further comprise one or more exemplary breakpoints, e.g., breakpoint 134, disposed on elongated shaft 104, further apart from collector tip 106. In an exemplary embodiment, elongated shaft 104 may be safely and easily break off at breakpoint 134 resulting in separation of collector tip 106 from elongated shaft 104. In an exemplary embodiment, “breakpoint 134” may refer to a site/point on elongated shaft's 104 body, disposed on elongated shaft 130, on a rear side of collector tip 106, where an exemplary body of elongated shaft 104 is smaller in width (compared to the rest of elongated shaft's 104 body), i.e., may be reduced in thickness, such that elongated shaft 104 may be easily broken off (for example by bending elongated shaft 104 at breakpoint 134).

In an exemplary embodiment, collector tip 106 may be available in different shapes, sizes, and materials for different applications. Collector tip 106 may made of different materials including, but not limited to, rayon, nylon, polyester, cotton, polyurethane, calcium alginate, and other synthetic, natural, and composite materials. Meanwhile, in one or more exemplary embodiments, collector tip 106 may be manufactured in different shapes including, but not limited to, spoon shaped, paddle shaped, brush shaped, etc. In an exemplary embodiment, hollow cover 116 may be made of a resilient material, such as plastic. In an exemplary embodiment, hollow cover 116′s shape may correspond to shape of collector tip 106 and elongated shaft 104. In an exemplary embodiment, exemplary shieldable swab assembly 100 may be manufactured in different dimensions to enable its easy movement to different target sites. In an exemplary embodiment, elongated shaft 104 of shieldable swab assembly 100 may comprise of different materials, e.g., plastic, silicon, etc.

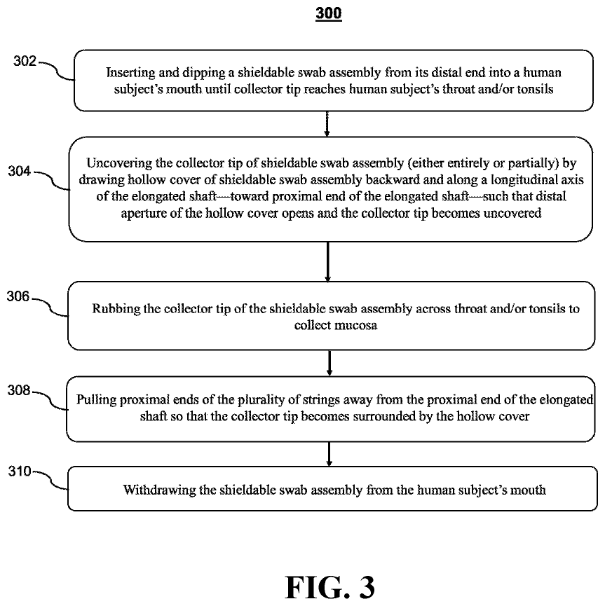

In an exemplary embodiment, exemplary shieldable swab assembly 100 may be used for rectal wall sampling, oropharyngeal sampling, nasopharyngeal sampling, nasal sampling, oral sampling, vagina sampling, laryngeal sampling, etc. FIG. 3 illustrates exemplary method 300 for sampling oropharyngeal mucosa using exemplary shieldable swab assembly 100, consistent with one or more exemplary embodiments of the present disclosure. In an exemplary embodiment, exemplary method 300 may comprise: inserting and dipping shieldable swab assembly 100 from its distal end 108 into a human subject's mouth until collector tip 106 reaches human subject's throat and/or tonsils (step 302); uncovering collector tip 106 of shieldable swab assembly 100 (either entirely or partially) by drawing hollow cover 116 of shieldable swab assembly 100 backward and along longitudinal axis 112 of elongated shaft 104—toward proximal end 110 of elongated shaft 104—such that distal aperture 123 of hollow cover 116 opens and collector tip 106 becomes uncovered (step 304); rubbing collector tip 106 of shieldable swab assembly 100 across throat and/or tonsils to collect mucosa (step 306); pulling proximal ends 128 of plurality of strings 118 away from proximal end 110 of elongated shaft 104 so that collector tip 106 becomes surrounded by hollow cover 116 (step 308); and withdrawing exemplary shieldable swab assembly 100 from human subject's mouth (step 310). In an exemplary embodiment, exemplary method 100 may be accomplished by a pathology collector.

In further detail with respect to step 302, step 302 may comprise inserting and dipping shieldable swab assembly 100 from its distal end 108 into a human subject's mouth until collector tip 106 reaches human subject's throat and/or tonsils. In an exemplary embodiment, exemplary shieldable swab assembly 100 may be pre-sterilized and packed in a disposable package to be protected from environmental contaminations. In an exemplary embodiment, inserting and dipping exemplary shieldable swab assembly 100 from its distal end 108 into a human subject's mouth until collector tip 106 reaches human subject's throat and/or tonsils may include inserting and dipping exemplary shieldable swab assembly 100, from its distal end 108 into a human subject's mouth by holding handle 132 of exemplary shieldable swab assembly 100 between fingers (e.g., between index finger and thumb).

Step 304 may include uncovering collector tip 106 of shieldable swab assembly 100 (either entirely or partially) by drawing hollow cover 116 of shieldable swab assembly 100 backward and along longitudinal axis 112 of elongated shaft 104—toward proximal end 110 of elongated shaft 104—such that distal aperture 123 of hollow cover 116 opens and collector tip 106 becomes uncovered. In an exemplary embodiment, uncovering collector tip 106 of exemplary shieldable swab assembly 100 (either entirely or partially) may comprise an exemplary process including: i) holding shieldable swab assembly 100 with one hand and gripping hollow cover 116 or band 124 with the other hand; and ii) drawing/sliding back hollow cover 116 such that an entire or at least a portion of collector tip 106 becomes uncovered/exposed.

Step 306 may include rubbing collector tip 106 of shieldable swab assembly 100 across throat and/or tonsils to collect mucosa. In an exemplary embodiment, rubbing collector tip 106 of shieldable swab assembly 100 across throat and/or tonsils to collect mucosa may include gently rubbing collector tip 106 against both tonsils and posterior pharynx. In an exemplary embodiment, gently rubbing collector tip 106 against both tonsils and posterior pharynx may include gently rubbing collector tip 106 against both tonsils and posterior pharynx while pressing human subject's tongue down using a tongue depressor.

Step 308 may include pulling proximal ends 128 of plurality of strings 118 away from proximal end 110 of elongated shaft 104 so that collector tip 106 becomes surrounded by hollow cover 116. In an exemplary embodiment, pulling proximal ends 128 of plurality of strings 118 away from proximal end 110 of elongated shaft 104 so that collector tip 106 becomes surrounded by hollow cover 116 may comprise an exemplary process including: i) gripping knot 129 and/or proximal ends 128 of plurality of strings 118; and ii) pulling knot 129 and/or proximal ends 128 of plurality of strings 118 away from proximal end 110 of elongated shaft 104. In an exemplary embodiment, in step 308, hollow cover 116 may adopt third configuration 210 (shown and explained in FIG. 2B). In an exemplary embodiment, pulling proximal ends 128 of plurality of strings 118 away from proximal end 110 of elongated shaft 104 may include pulling proximal ends 128 of plurality of strings 118 away until distal aperture 123 of hollow cover 116 may become closed around distal end 131 (see FIG. 2B) of collector tip 106 and distal end 120 of hollow cover 116 becomes squeezed and fitted around collector tip 106.

Step 310 may include withdrawing shieldable swab assembly 100 from human subject's mouth. In an exemplary embodiment, during step 310, hollow cover 116 may protect collector tip 106 from touching neighboring/surrounding sites around the human subject's throat and tonsils, e.g., tongue, buccal mucosa, teeth, palate, saliva, gingiva, etc. Contact of collector tip 106 to surrounding sites of a target site during swab-sampling may result in erroneous analytical test results. In an exemplary embodiment, after withdrawing shieldable swab assembly 100, a pathology collector may again uncover collector tip 106—by gripping hollow cover 116 and drawing/sliding hollow cover 116 backward along longitudinal axis 112—and bend elongated shaft 104 at breakpoint 134 to break elongated shaft 104 and release/separate collector tip 106 (for example in a tube).

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. An element proceeded by “a” or “an” does not, without further constraints, preclude the existence of additional identical elements in the process, method, article, or apparatus that comprises the element.

Unless otherwise stated, all measurements, values, ratings, positions, magnitudes, sizes, and other specifications that are set forth in this specification, are approximate, not exact. They are intended to have a reasonable range that is consistent with the functions to which they relate and with what is customary in the art to which they pertain.

It will be understood that the terms and expressions used herein have the ordinary meaning as is accorded to such terms and expressions with respect to their corresponding respective areas of inquiry and study, except where specific meanings have otherwise been set forth herein. Relational terms such as “first” and “second” and the like may be used solely to distinguish one entity or action from another without necessarily requiring or implying any actual such relationship or order between such entities or actions.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A shieldable swab assembly, comprising:
an elongated shaft comprising:
a distal end comprising a distal opening;
a proximal end comprising a proximal opening; and
a central duct extending through the elongated shaft from the distal end of the elongated shaft to the proximal end of the elongated shaft;
a collector tip secured around the distal end of the elongated shaft; and
a slidable cover assembly carried by the elongated shaft and configured to removably enclose the collector tip, the slidable cover assembly comprising:
a hollow cover made of a resilient material, the hollow cover comprising:
a proximal end comprising a proximal aperture, the proximal end of the hollow cover slidably fastened around the elongated shaft on a rear side of the collector tip; and
a distal end comprising a distal aperture, wherein the distal aperture of the hollow cover adopts a closed configuration and an open configuration;
wherein the elongated shaft passes through the proximal and distal apertures of the hollow cover, the hollow cover capable of moving along a longitudinal axis of the elongated shaft toward the proximal and distal ends of the elongated shaft; and
a plurality of strings extending throughout the central duct of the elongated shaft, each respective string of the plurality of strings comprising:
a distal end extending outwardly from the distal opening of the elongated shaft, the distal end of the each respective string attached to an outer surface of the distal end of the hollow cover, wherein distal ends of the plurality of strings are arranged radially and concentrically around the distal end of the hollow cover; and
a proximal end extending outwardly from the proximal opening of the elongated shaft, wherein proximal ends of the plurality of strings are tied to one another;
wherein the hollow cover is configured to adopt a plurality of configurations comprising:
a first configuration in a first state when the hollow cover encloses the collector tip and the distal aperture of the hollow cover adopts said closed configuration in which the distal aperture of the hollow cover is closed around a distal end of the collector tip;
a second configuration in a second state when the hollow cover is moved along the longitudinal axis of the elongated shaft toward the proximal end of the elongated shaft such that the distal aperture of the hollow cover opens and the collector tip or a portion of the collector tip becomes uncovered; and
a third configuration in a third state when the proximal ends of the plurality of strings are pulled away from the proximal end of the elongated shaft such that the hollow cover when in the second state is retracted toward the distal end of the elongated shaft and encloses the collector tip.

2. The shieldable swab assembly of claim 1, wherein in the third state, the plurality of strings pulled away from the proximal end of the elongated shaft comprises the proximal ends of the plurality of strings pulled away from the proximal end of the elongated shaft so that the hollow cover is retracted toward the distal end, and the collector tip is enclosed, and the distal aperture of the hollow cover is closed around the distal end of the collector tip.

3. The shieldable swab assembly of claim 1, wherein the distal end of the hollow cover is squeezed and fitted around the distal end of the collector tip when the hollow cover is in the first state.

4. The shieldable swab assembly of claim 1, wherein the proximal end of the hollow cover is slidably fastened around the elongated shaft using a band twisted, perpendicular to the longitudinal axis of the elongated shaft, around the proximal end of the hollow cover.

5. The shieldable swab assembly of claim 4, wherein the band is configured to fasten the proximal end of the hollow cover around the elongated shaft such that the hollow cover is capable of sliding on the elongated shaft along the longitudinal axis of the elongated shaft and toward the proximal and distal ends of the elongated shaft.

6. The shieldable swab assembly of claim 4, wherein the band is an elastic band.

7. The shieldable swab assembly of claim 1, wherein the elongated shaft further comprises a stopper mounted on the elongated shaft, further apart from the proximal end of the elongated shaft and on a rear side of the proximal end of the hollow cover.

8. The shieldable swab assembly of claim 7, wherein the stopper is configured to prevent the hollow cover from sliding up to the proximal end of the elongated shaft and from being removed from the elongated shaft.

9. The shieldable swab assembly of claim 1, wherein the hollow cover is made of plastic.

10. A shieldable swab assembly, comprising:
an elongated shaft comprising:
a distal end comprising a distal opening;
a proximal end comprising a proximal opening; and
a central duct extending through the elongated shaft from the distal end of the elongated shaft to the proximal end of the elongated shaft;
a collector tip secured around the distal end of the elongated shaft; and
a slidable cover assembly carried by the elongated shaft and configured to removably enclose the collector tip, the slidable cover assembly comprising:
a hollow cover made of a resilient material, the hollow cover comprising:
a proximal end comprising a proximal aperture, the proximal end of the hollow cover slidably fastened around the elongated shaft on a rear side of the collector tip; and
a distal end comprising a distal aperture, wherein the distal aperture of the hollow cover adopts a closed configuration and an open configuration;
wherein the elongated shaft passes through the proximal and distal apertures of the hollow cover, the hollow cover capable of moving along a longitudinal axis of the elongated shaft toward the proximal and distal ends of the elongated shaft, wherein the proximal end of the hollow cover is slidably fastened around the elongated shaft using a band twisted perpendicular to the longitudinal axis of the elongated shaft around the proximal end of the hollow cover, the band configured to fasten the proximal end of the hollow cover around the elongated shaft such that the hollow cover is capable of sliding on the elongated shaft along the longitudinal axis of the elongated shaft and toward the proximal and distal ends of the elongated shaft; and
a plurality of strings extending throughout the central duct of the elongated shaft, each respective string of the plurality of strings comprising:
a distal end extending outwardly from the distal opening of the elongated shaft, the distal end of the each respective string attached to an outer surface of the distal end of the hollow cover, wherein distal ends of the plurality of strings are arranged radially and concentrically around the distal end of the hollow cover; and
a proximal end extending outwardly from the proximal opening of the elongated shaft, wherein proximal ends of the plurality of strings are tied to one another.

11. The shieldable swab assembly of claim 10, wherein the hollow cover is configured to adopt a plurality of configurations comprising:
a first configuration in a first state when the hollow cover encloses the collector tip and the distal aperture of the hollow cover adopts said closed configuration in which the distal aperture of the hollow cover is closed around a distal end of the collector tip;
a second configuration in a second state when the hollow cover is moved along the longitudinal axis of the elongated shaft toward the proximal end of the elongated shaft such that the distal aperture of the hollow cover opens and the collector tip or a portion of the collector tip becomes uncovered; and
a third configuration in a third state when the proximal ends of the plurality of strings are pulled away from the proximal end of the elongated shaft such that the hollow cover when in the second state is retracted toward the distal end of the elongated shaft and encloses the collector tip.
